(19) United States
(12) Patent Application Publication
(10) Pub. No.: US 2019/0298721 A1
(43) Pub. Date: Oct. 3, 2019

(54) STABLE PRO-VITAMIN DERIVATIVE COMPOUNDS, PHARMACEUTICAL AND DIETARY COMPOSITIONS, AND METHODS OF THEIR USE

(71) Applicant: CORNELL UNIVERSITY, Ithaca, NY (US)

(72) Inventors: Patrick J. STOVER, Ithaca, NY (US); Martha S. FIELD, Burdett, NY (US)

(21) Appl. No.: 16/315,729

(22) PCT Filed: Jul. 6, 2017
(86) PCT No.: PCT/US17/40898

§ 371 Date: Jan. 7, 2019

Related U.S. Application Data
(60) Provisional application No. 62/359,040, filed on Jul. 6, 2016.

(51) Int. Cl. A61K 31/519; C07D 475/04; A61K 45/06; A61K 9/00; A23L 33/15

(52) U.S. Cl. CPC A61K 31/519; C07D 475/04; A61K 45/06; A23V 2002/00; A61K 9/0029; A23L 33/15; A61K 9/0053

(57) ABSTRACT

The present invention relates to a compound of Formula I: [Formula should be inserted here], where R₁, R₂, and n are as described above, and pharmaceutically acceptable salts and solvates thereof. The present invention also relates to use of the above-noted compounds and compositions including those compounds.

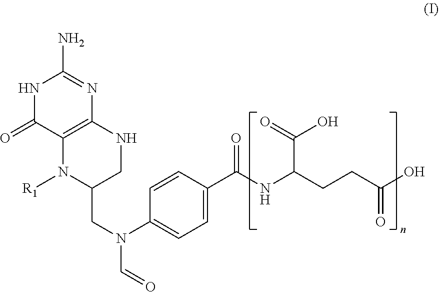

This application claims the benefit of U.S. Provisional Patent Application Ser. No. 62/359,040, filed Jul. 6, 2016, which is hereby incorporated by reference in its entirety.

This invention was made with government support under grant number R37DK58144 awarded by the National Institutes of Health. The government has certain rights in this invention.

### FIELD OF THE INVENTION

The present invention relates to compositions including stable derivatives of tetrahydrofolate and methods of using the same.

### BACKGROUND

Folate is a B-vitamin found in food including fruits and vegetables and is essential for human health and physiological function (Stover, “Physiology of Folate and Vitamin B12 in Health and Disease,” Nutr. Rev. 62(6 Pt 2):53-12 (2004)). It is a metabolic cofactor required for the synthesis of nucleotides for DNA synthesis and for synthesizing the amino acid methionine from homocysteine which is required for protein synthesis and methylation reactions. Low dietary folate intake, poor nutritional status, or impairments in folate utilization are associated with numerous pathologies including birth defects, anemia, cancer, and neurodegenerative conditions.

Folate is chemically unstable and oxidizes readily, especially when food is cooked. The primary folate present is (6)-5-methyl-tetrahydrofolate (5-methyl-THF), which is also a commercial supplement and medical food that is not widely used. Folic acid is a chemically stable and synthetic form of folate that is not normally found in nature, but is able to be converted to natural folate in living cells by chemical reduction through the NADPH-requiring enzyme dihydrofolate reductase (DHFR). Folic acid nutritional supplements are commonly administered routinely to women as part of a prenatal vitamin to prevent birth defects, and is present in many multivitamin pills and nutritional supplements.

Folic acid is not effective in treating all chronic disease, especially brain diseases associated with brain-specific nutritional deficiency due to loss of blood-brain barrier function. Patients with low cerebral spinal fluid (CSF) folate require very high oral doses (up to 20 mg/kg/day) of reduced folate ((6S)-5-formyl-THF; otherwise known as Leucovorin) or ((6S)-5-methyl-THF) to normalize CSF folate levels (Quijada-Fraile et al., “Follow-up of Folinic Acid Supplementation for Patients with Cerebral Folate Deficiency and Kearns-Sayre Syndrome,” Orphanet J. Rare Dis. 9:217 (2014); Molero-Luis et al., “Clinical, Etiological and Therapeutic Aspects of Cerebral Folate Deficiency,” Expert Rev. Neurother. 15(7):793-802 (2015); Pineda et al., “Cerebral Folate Deficiency and Leukoencephalopathy Caused by a Mitochondrial DNA Deletion,” Ann. Neurol. 59(2):394-398 (2006); Stover et al., “Folate Nutrition and Blood-Brain Barrier Dysfunction,” Curr. Opin. Biotechnol. 44:146-152 (2017)). Folic acid at high doses is not deemed beneficial and is not recommended in many cases of cerebral folate deficiency. This is due to both low rates of folic acid transport across the blood-brain barrier and low brain levels of DHFR, the enzyme required to convert folic acid into a biological and functional cofactor (Stover et al., “Folate Nutrition and Blood-Brain Barrier Dysfunction,” Curr. Opin. Biotechnol. 44:146-152 (2017)).

The present invention is directed to overcoming deficiencies in the art.

### SUMMARY OF THE INVENTION

One aspect of the present invention relates to a compound of Formula I:

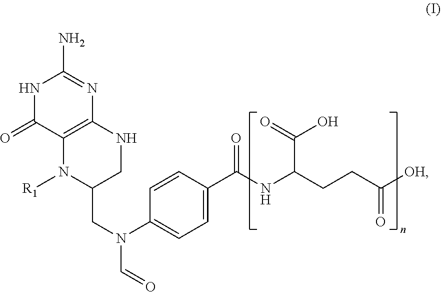

where

R₁ is a C₁-C₆ alkyl or

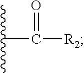

R₂ is H or C₁-C₆ alkyl; and

n is 1 to 8,

or a pharmaceutically acceptable salt thereof, or a solvate thereof.

Another aspect of the present invention relates to a pharmaceutical or dietary composition comprising the compound of Formula I according to the present invention and a pharmaceutically or dietetically suitable carrier.

Another aspect of the present invention relates to a method of supplementing the dietary needs of a subject. This method involves administering to the subject a dietary supplementing effective amount of the compound of Formula I according to the present invention.

Another aspect of the present invention relates to a method of increasing levels of (6S)-THF in a subject. This method involves selecting a subject and administering to the selected subject an effective amount of the compound of Formula I according to the present invention to increase levels of (6S)-THF in the selected subject.

### BRIEF DESCRIPTION OF THE DRAWINGS

FIGS. 1A-G are graphs showing HPLC (FIGS. 1A-B), positive ion mode HPLC-MS/MS spectra (FIGS. 1C-F), and NMR spectra (FIG. 1G) of (6S)-5-formyl, 10-formylTHF. FIGS. 1C and 1D show representative chromatograms. FIG. 1E shows mass spectra of the parent ion. FIG. 1F shows ion fragments. NMR spectra was taken from unpurified reaction.

FIG. 2 is a graph showing an HPLC trace of (6S)-5-methyl, 10-formyl-THF indicating greater than 90% purity. Major peak is 5-methyl, 10-formyl-THF. The chromatogram was taken at 260 nm wavelength.

FIGS. 3A-B show UV spectrum of 5-methyl, 10-formyl-THF.

FIG. 4 is an NMR spectrum of 5-methyl, 10-formyl-THF.

FIGS. 5A-D show positive ion mode HPLC-MS/MS spectra of (6S) 5-methyl, 10-formyl-THF. FIGS. 5A-B show representative chromatograms. FIG. 5C shows parent ion. FIG. 5D shows ion fragments.

FIGS. 6A-C are graphs showing proliferation of HeLa cells (FIGS. 6A and 6C) and immortalized mouse fibroblasts (FIG. 6B) cultured in Defined αMEM supplemented with varying concentrations of folic acid, (6S)-Leucovorin, (6S)-5-methyl, 10-formyl-THF, and (6S) 5-formyl, 10-formylTHF. Proliferation rates were determined using brightfield image analysis. Both (6S)-5-methyl, 10-formyl-THF and (6S) 5-formyl, 10-formylTHF support cell growth in a similar dose-response as (6S)-Leucovorin and are superior to folic acid.

FIG. 7 is a bar graph showing intracellular accumulation of folate over 72 hours following exposure to either (6S)-5-methyl, 10-formyl-THF or (6S)-Leucovorin. Data are shown as average and standard deviation of 5 technical replicates per experimental condition. Order of the bar graphs left to right is as written in the legend under the X axis. FA: Folic Acid. 5CH3THF: (6S) 5-methyltetrahydrofolate. 5CHOTHF: Leucovorin or (6S)-5-formyltetrahydrofolate. Declan: (6S) 5-methyl, 10-formyltetrahydrofolate. Brainfol: (6S) 5-formyl, 10-formyltetrahydrofolate.

FIG. 8 is a graph showing chemical stability of folic acid (FA), (6S) 5-methyltetrahydrofolate (5CH3THF). (6S)-Leucovorin (5CHOTHF, (6S)-5-formyltetrahydrofolate), (6S) 5-methyl, 10-formyltetrahydrofolate (Declan), or (6S) 5-formyl, 10-formyltetrahydrofolate (Brainfol). (6S) 5-methyl, 10-formyltetrahydrofolate (Declan), or (6S) 5-formyl, 10-formyltetrahydrofolate (Brainfol) demonstrate increased chemical stability relative to (6S) 5-methyltetrahydrofolate (5CH3THF).

FIGS. 9A-B are graphs showing stability of (6)-5-formyl, 10-formyl-THF and folic acid in AIN93G mouse food pellets. Data are shown as average and standard deviation of 3 technical replicates per time point. FIG. 9A shows the stability of (6)-5-formyl, 10-formyl-THF and folic acid in AIN93G mouse food pellets stored at room temperature. FIG. 9B shows the stability of (6)-5-formyl, 10-formyl-THF and folic acid in AIN93G mouse food pellets stored at −20° C. Mice have been fed the AIN-93G diet containing (6S)-5-formyl, 10-formyl-THF for 8 weeks without any adverse symptoms.

### DETAILED DESCRIPTION OF THE INVENTION

In the following description, reference is made to the accompanying drawings that form a part hereof, and in which is shown by way of illustration specific embodiments which may be practiced. These embodiments are described in detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

As discussed throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “alkyl” means an aliphatic hydrocarbon group which may be straight or branched. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, 3-pentyl, and the like.

One aspect of the present invention relates to a compound that is a synthetic form of reduced folate and that is pro-vitamin form of folate. The compound is that of Formula I:

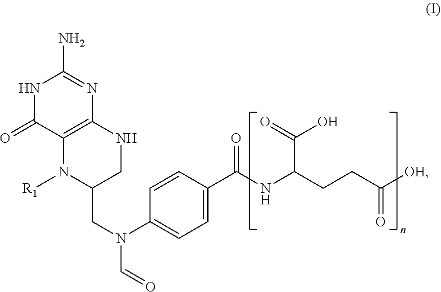

where

R₁ is a C₁-C₆ alkyl or

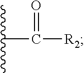

R₂ is H or C₁-C₆ alkyl; and

n is 1 to 8,

or a pharmaceutically acceptable salt thereof, or a solvate thereof.

In one embodiment, R₁ is methyl.

In another embodiment, R₁ is formyl (i.e., R₁ is

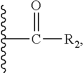

where R₂ is H.

In one embodiment, the compound is (6S)-5-methyl, 10-formylTHF (previously referred to as ‘Declan’) having the following structure:

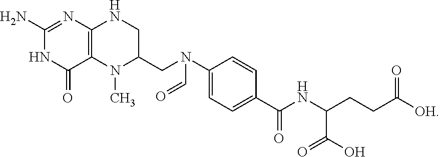 [SMILES: CN1c2c(nc(N)[nH]c2=O)NCC1CN(C=O)c1ccc(C(=O)NC(CCC(=O)O)C(=O)O)cc1]

(6S)-5-Methyl, 10-formylTHF is an N-10 formylated form of (6S)-5-methylTHF. This compound name may also be written as (6S)-5-methyl, 10-formyl-tetrahydrofolate or (6S)-5-methyl, 10-formyltetrahydrofolate. (6S)-5-Methyl, 10-formylTHF is more stable than (6S)-5-methylTHF.

In another embodiment, the compound is (6S)-5-formyl, 10-formyltetrahydrofolate (previously referred to as ‘Brainfol’), having the following structure:

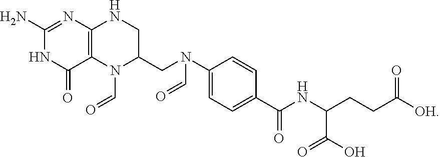 [SMILES: Nc1nc2c(c(=O)[nH]1)N(C=O)C(CN(C=O)c1ccc(C(=O)NC(CCC(=O)O)C(=O)O)cc1)CN2]

(6S)-5-Formyl, 10-formylTHF is an N-10 formylated form of (6S)-5-formylTHF (Leucovorin). This compound name may also be written as (6S)-5-formyl, 10-formyl-tetrahydrofolate or (6S)-5-formyl, 10-formyltetrahydrofolate. (6S)-5-Formyl, 10-formylTHF is more stable than (6S) 5-formylTHF.

Each of (6S)-5-methyl, 10-formylTHF and (6S)-5-formyl, 10-formylTHF is converted to biologically active (6S)-tetrahydrofolate (THF) once it is transported into the cells and is chemically identical to the natural vitamin.

Both (6S)-5-methyl, 10-formylTHF and (6S)-5-formyl, 10-formylTHF have chemical stability characteristics similar to that of folic acid, but are like folate in that they are chemically reduced. To be converted from their pro-vitamin form, only a single formyl group has to be removed from the N-10 position. Thus, they do not exhibit the toxic effects of folic acid as they are structurally similar to natural food folate. The enzyme that removes the N-10 formyl group to activate (6S)-5-methyl, 10-formylTHF and (6S)-5-formyl, 10-formylTHF-10-formyltetrahydrofolate dehydrogenase—is not expressed or is expressed at very low levels in tumor cells.

(6S)-5-Methyl, 10-formylTHF is more stable than (6S)-5-methylTHF, and is converted to biologically active (6S)-5-methylTHF once it is transported into the cells, where it is then chemically identical to the natural folate vitamin. Lower doses of (6S)-5-methyl, 10-formylTHF are required to address nutritional deficiencies, including tissue-specific nutritional deficiencies, than are currently addressed through (6S)-5-methylTHF treatment.

(6S)-5-Formyl, 10-formylTHF is more stable than Leucovorin Acid ((6S)-5-formylTHF), and is converted to biologically active (6S)-5-formylTHF once it is transported into the cells, again making it chemically identical to the natural vitamin. Lower doses of (6S)-5-formyl, 10-formylTHF are required to address brain and other nutritional deficiencies, including tissue-specific nutritional deficiencies, than are currently addressed through oral (6S)-5-methylTHF supplementation. Furthermore, (6S)-5-formyl, 10-formylTHF metabolism does not require vitamin B12 and, therefore, its processing into the intracellular folate pools occurs independent of vitamin B12 deficiency. Unlike Leucovorin acid (6S) 5-formylTHF), (6S)-5-formyl, 10-formylTHF is stable in acid because it does not convert to 5,10-methenylTHF in acid and, therefore, can be taken orally and does not require parenteral administration as is typical for Leucovorin acid.

(6S)-5-Methyl, 10-formylTHF and (6S)-5-formyl, 10-formylTHF, as opposed to folic acid, enter cells differently through selective transport mechanisms. Reduced folates are transported into cells through the reduced folate carrier, whereas folic acid binds tightly to the folate receptor (FRα), a glycoprotein that is anchored to the cell membrane of epithelial cells and cancer cells, and transports folic acid into cells by receptor-mediated endocytosis. For these reasons, (6S)-5-methyl, 10-formylTHF and (6S)-5-formyl, 10-formylTHF are more effectively transported into non-cancerous cells compared to cancerous cells.

(6S)-5-Methyl, 10-formylTHF and (6S) 5-formyl, 10-formylTHF are the only pro-vitamin forms of reduced folate ever reported. They are more stable than the natural forms of folate, (6S)-5-methylTHF and Leucovorin ((6S)-5-formylTHF), which are commonly administered orally and/or parenterally as vitamins or as medical foods. (6S)-5-Formyl, 10-formylTHF is stable in acid unlike (6S)-5-methylTHF and Leucovorin ((6S) 5-formylTHF) is unstable in acid and, therefore, it can be administered orally. (6S)-5-Methyl, 10-formylTHF and (6S)-5-formyl, 10-formylTHF are transported into cells with different efficiencies than folic acid.

Cancer cells downregulate the (6S)-5-methyl, 10-formylTHF and (6S)-5-formyl, 10-formylTHF activating enzyme, 10-formyltetrahydrofolate dehydrogenase and, therefore, cancer cells may be less efficient at catalyzing the bio-conversion of these compounds to their functional form in cancer cells. Therefore, they may be less effective than folic acid and natural folate in promoting tumor growth.

In one embodiment, compounds of the present invention are monoglutamyl-, and/or polyglutamyl-derivatives of any compound of Formula I, and salts (e.g., Ca or Na₂ salts) of any of these compounds. As will be understood by a person of ordinary skill in the art, polyglutamyl-derivatives include those compounds of Formula I where n>1.

In another embodiment, compounds of the present invention are the prodrugs, the pharmaceutically acceptable salts, the oxides, the solvates (e.g., hydrates), and inclusion complexes of Formula I compounds, where the context so permits, as well as any stereoisomeric forms, or a mixture of any such forms of that compound in any ratio.

Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed by the present invention. In accordance with some embodiments, a compound as described herein, including in the contexts of pharmaceutical compositions, methods of treatment, and compounds per se, is provided as the salt form. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

In a further embodiment, compounds of the present invention are various salt forms (e.g., calcium salt, sodium salt, etc.) of the compound of Formula I.

Another aspect of the present invention relates to methods of synthesizing the compounds according to the present invention. Exemplary synthesis is described in the examples infra.

In one embodiment, compounds of the present invention where n is 1 can be prepared according to the following general scheme:

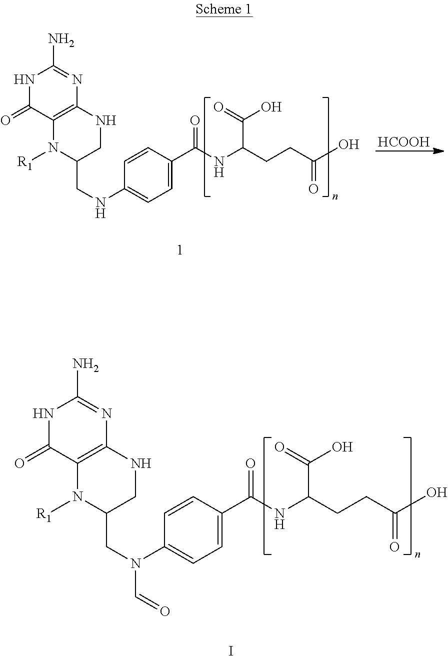

Reaction of the carboxylic acid derivative (1) with formic acid leads to formation of the compound of Formula I. This reaction can be carried out at room temperature or at elevated temperatures. It can be carried out at a temperature of 0° C. to 200° C., at a temperature of 20° C. to 150° C., or at a temperature of 50° C. to 70° C.

In the cell, folate derivatives are modified with a poly-gamma-glutamate polypeptide, and polyglutamate versions of these compounds could be synthesized for laboratory-based scientific experiments.

A further aspect of the present invention relates to a pharmaceutical or dietary composition comprising one or more compounds according to the present invention. In one embodiment, the composition includes a pharmaceutically or dietetically suitable carrier.

The compounds of the present invention may be provided as a composition with a pharmaceutically or dietetically acceptable carrier. Such compositions may include physiologically acceptable carriers that are inherently non-toxic and non-therapeutic. Examples of such carriers include vegetable proteins, soy proteins, ion exchangers, soft gels, oils, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms include polysaccharides such as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

In accordance with the present invention, the compound of Formula I (i.e., compounds according to the present invention) may be used to formulate pharmaceuticals, nutraceuticals, botanical drugs, herbal medicines, food additives, functional foods, medical foods, nutrition products, cosmetics, beverages, and the like.

Accordingly, in one embodiment of the present invention, the compound or composition is part of a nutraceutical composition, pharmaceutical composition, functional food, functional nutrition product, medical food, medical nutrition product, or dietary supplement.

In certain embodiments, compounds or compositions of the present invention may be combined with herbal medicines. In other embodiments, the compounds or compositions of the present invention may be formulated as botanical drugs. A botanical drug is a product that includes vegetable materials, which may include plant materials, algae, macroscopic fungi, or combinations thereof, which is intended for use in the diagnosis, cure, mitigation, treatment, or prevention of disease in humans. In certain embodiments, the botanical drug product may be available as (but not limited to) a solution (e.g., tea), powder, tablet, capsule, elixir, topical cream or gel, or injection.

In certain embodiments, compounds or compositions of the present invention may be formulated as nutraceuticals. Nutraceutical formulations of interest include foods for veterinary or human use, including health food bars, drinks and drink supplements, and the like. These foods are enhanced by the inclusion of a composition of the present invention. For example, in the treatment of an intestinal tumor, the normal diet of a patient may be supplemented by a nutraceutical formulation taken on a regular basis. Such nutraceuticals may or may not contain calories. The term nutraceutical composition as used herein include food products, foodstuff, dietary supplements, nutritional supplements, or a supplement compositions for a food product or a foodstuff. Thus, in another embodiment, the present invention relates to a nutraceutical, where the nutraceutical is a food product, foodstuff, dietary supplement, nutritional supplement, or a supplement composition for a food product or a foodstuff.

As used herein, the term “food product” refers to any food or feed suitable for consumption by humans or animals. The food product may be a prepared and packaged food (e.g., mayonnaise, salad dressing, bread, or cheese) or an animal feed (e.g., extruded and pelleted animal feed, coarse mixed feed, or pet food composition). As used herein, the term “foodstuff” refers to any substance fit for human or animal consumption. Food products or foodstuffs include beverages such as non-alcoholic and alcoholic drinks as well as liquid preparation to be added to drinking water and liquid food. Non-alcoholic drinks include, without limitation, soft drinks, sport drinks, fruit juices, such as for example orange juice, apple juice and grapefruit juice; lemonades, teas, near-water drinks, milk, milk replacements, baby formulas, and other dairy drinks, such as, for example, yogurt drinks and diet drinks.

In another embodiment, food products or foodstuffs refer to solid or semi-solid foods comprising compounds or compositions of the present invention. These forms can include, but are not limited to, baked goods such as bars, cakes, cookies, puddings, dairy products, confections, snack foods, or frozen confections or novelties (e.g., ice cream, milk shakes), prepared frozen meals, candy, snack products (e.g., chips), liquid food such as soups, spreads, sauces, salad dressings, prepared meat products, cheese, yogurt, and any other fat or oil containing foods and food ingredients (e.g., wheat flour). The term food products or foodstuffs also include functional foods and prepared food products, the latter referring to any pre-packaged food approved for human consumption. The term functional food or functional nutrition product refers to a food or nutrition product that is sold (e.g., in a supermarket or online) without any restrictions. The term medical food or medical nutrition product refers to a food or nutrition product which is prescribed by a physician.

In certain embodiments, compounds or compositions of the present invention may be formulated as dietary supplements. Dietary supplements of the present invention may be delivered in any suitable format. In certain embodiments, dietary supplements are formulated for oral delivery. The ingredients of the dietary supplement of this invention are contained in acceptable excipients and/or carriers for oral consumption. The carrier may be a liquid, gel, gelcap, capsule, powder, solid tablet (coated or non-coated), tea, or the like. The dietary supplement is preferably in the form of a tablet or capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). The various ingredients and the excipient and/or carrier are mixed and formed into the desired form using conventional techniques. The tablet or capsule may be coated with an enteric coating that dissolves at a pH of about 6.0 to 7.0. A suitable enteric coating that dissolves in the small intestine but not in the stomach is cellulose acetate phthalate.

In other embodiments, the dietary supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food (e.g., enclosed in caps of food or beverage containers for release immediately before consumption). In one embodiment, the dietary supplement is a baby formula powder to be mixed or emulsified in a liquid, e.g., water.

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like.

In other embodiments, the present invention provides nutritional supplements (e.g., energy bars or meal replacement bars or beverages) comprising compounds or compositions of the present invention. The nutritional supplement may serve as meal or snack replacement and generally provide nutrient calories. However, as noted above, supplements which do not contain calories may also be used. In certain embodiments, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, where simple carbohydrates are mono- or disaccharides. The nutritional supplement contains, in one embodiment, combinations of sources of carbohydrate of three levels of chain length (simple, medium, and complex (e.g., sucrose, maltodextrins, and uncooked cornstarch)).

Sources of protein to be incorporated into the nutritional supplement can be any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein, and hydrolysates or mixtures thereof. Soy protein has an almost perfect Protein Digestibility Corrected Amino Acid Score (PDCAAS) and by this criterion soy protein is the nutritional equivalent of meat and eggs for human growth and health. These proteins have high biological value (i.e., a high proportion of the essential amino acids). See MODERN NUTRITION IN HEALTH AND DISEASE (Lea & Febiger, 8th ed. 1986), which is hereby incorporated by reference in its entirety.

The nutritional supplement can also contain other ingredients, such as one or a combination of other vitamins, minerals, antioxidants (e.g., vitamin C, vitamin E, 3 carotene, Ubichinone 10, and vitamin A), fiber, and other dietary supplements (e.g., protein, amino acids, choline, lecithin, omega-3 fatty acids, and others discussed herein). Selection of one or several of these ingredients is a matter of formulation, design, consumer preference, and end-user. The amounts of these ingredients added to the dietary supplements of the invention are readily known to the skilled artisan. In one embodiment, vitamins and/or minerals are added, as described below.

The nutritional supplement can be provided in a variety of forms, and by a variety of production methods. In one embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

As would be understood by those of skill in the art, other ingredients can be added to those described herein, for example, fillers, emulsifiers, and preservatives for the processing or manufacture of a nutritional supplement.

Additionally, flavors, coloring agents, spices, nuts, and the like may be incorporated into the formulations described herein. Flavorings can be in the form of flavored extracts, volatile oils, chocolate flavorings, peanut butter flavoring, cookie crumbs, crisp rice, vanilla, or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil.

Emulsifiers may be added for stability of the formulations. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. Preservatives may also be added to the formulations to extend product shelf life. For example, preservatives such as potassium sorbate, sodium sorbate, potassium benzoate, sodium benzoate, or calcium disodium EDTA are used.

In addition to the carbohydrates described above, the formulations can contain natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

Moreover, a multi-vitamin and mineral supplement may be added to the formulations or compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

In accordance with the present invention, compounds or compositions of the present invention may be prepared as pharmaceutical compositions, such as those which may be particularly useful for the treatment or prevention of folate deficiency, disease-induced folate deficiency, folate-deficiency related diseases (e.g., folate-deficiency related birth defects), and cancer (e.g., intestinal tumors), respectively. Such compositions comprise a compound of the present invention (e.g., (6S)-5-methyl, 10-formylTHF and/or (6S) 5-formyl, 10-formylTHF) or a pharmaceutically acceptable salt, biologically active metabolite, solvate, hydrate, prodrug, enantiomer or stereoisomer thereof, and a pharmaceutically acceptable carrier and/or excipient.

The compositions of the present invention may be administered by various means, depending on their intended use, as is well known in the art. For example, if compositions of the present invention are to be administered orally, they may be formulated as tablets, capsules, granules, powders, or syrups. Alternatively, formulations of the present invention may be administered parenterally as injections (intravenous, intramuscular, or subcutaneous), drop infusion preparations, or suppositories. For application by the ophthalmic mucous membrane route, compositions of the present invention may be formulated as eye drops or eye ointments. These formulations may be prepared by conventional means and, if desired, the compositions may be mixed with any conventional additive, such as an excipient, a binder, a disintegrating agent, a lubricant, a corrigent, a solubilizing agent, a suspension aid, an emulsifying agent, or a coating agent.

In formulations of the subject invention, wetting agents, emulsifiers, and lubricants, such as sodium lauryl sulfate and magnesium stearate, as well as coloring agents, release agents, coating agents, sweetening, flavoring and perfuming agents, preservatives, and antioxidants may be present in the formulated agents.

In one embodiment, the compound or composition according to the present invention is administered parenterally. In another embodiment of the present invention, the compound or composition according to the present invention is administered orally.

In one embodiment, the compound or composition according to the present invention is in capsule, tablet, granule, or lozenge form.

Subject formulations may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules, and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using excipients such as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups, and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics®, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

In certain embodiments, the compositions are formulated as a tablet, pill capsule, or other appropriate ingestible formulation, to provide a therapeutic dose in 10 ingestible formulations or fewer. In another example, a therapeutic dose is provided in 20, 15, 10, 5, 4, 3, or 2 ingestible formulations.

The compositions of the present invention may be in the form of a dispersible dry powder for pulmonary delivery. Dry powder compositions may be prepared by processes known in the art, such as lyophilization and jet milling, as disclosed in WO 91/16038 and as disclosed in U.S. Pat. No. 6,921,527, which are hereby incorporated by reference in their entirety. The composition of the present invention may be placed within a suitable dosage receptacle in an amount sufficient to provide a subject with a unit dosage treatment. The dosage receptacle may be one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. Nos. 4,227,522; 4,192,309; and 4,105,027, which are hereby incorporated by reference in their entirety.

A transdermal delivery system, transdermal patch, or patch refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for postmenopausal indications, and nicotine for smoking cessation. Patches suitable for use in the present invention include, but are not limited to, (1) the matrix patch; (2) the reservoir patch; (3) the multi-laminate drug-in-adhesive patch; and (4) the monolithic drug-in-adhesive patch (TRANSDERMAL AND TOPICAL DRUG DELIVER SYSTEMS (Ghosh et al. eds., 1997), which is hereby incorporated by reference in its entirety). These patches are well known in the art and generally available commercially.

The compositions of the present inventive subject matter may optionally contain any other vitamins, minerals, nutritional agents, therapeutic agents and the like. In one embodiment, the pharmaceutical or dietary composition includes one or more vitamins, one or more minerals, or combinations thereof.

These vitamins, minerals, and trace elements include, for example, Vitamin A or beta-carotene, Vitamin B1 (as Thiamin or Thiamin mononitrate), Vitamin B2 (as Riboflavin), Vitamin B3 (as Niacin), Vitamin B6 (as Pyridoxine or Pyridoxine hydrochloride), Vitamin B12 (cyanocobalamine), Vitamin H (Biotin), Vitamin C (Ascorbic Acid), Vitamin D, Vitamin E (as dl-Alpha Tocopherol Acetate), Vitamin K, Folacin, Niacinamide, Iron (as Ferrous Fumarate), Phosphorus, Pantothenic Acid (as Calcium Pantothenate), Iodine (as Potassium Iodide), Magnesium (as Magnesium Oxide), Zinc (as Zinc Oxide), Selenium (as Sodium Selenate), Copper (as Cupric Oxide), Manganese (as Manganese Sulfate), Chromium (as Chromium Chloride), Molybdenum (as Sodium Molybdate), Choline, Fluoride, Chloride, Potassium, Sodium, and mixtures thereof. Such vitamins, minerals and trace elements are commercially available from sources known by those of skill in the art. The dosage forms of the invention may be formulated using any pharmaceutically-acceptable forms of the vitamins and/or minerals described above, including their salts, which are known by those of skill in the art.

In one embodiment, the pharmaceutical or dietary composition according to the present invention includes one or more vitamins, minerals, trace elements, or combinations thereof selected from the group consisting of Vitamin A, Vitamin C, Vitamin D, Vitamin E, Vitamin K, Thiamin, Riboflavin, Niacin, Vitamin B12, Vitamin B6, Pantothenic Acid, Biotin, Choline, Chromium, Copper, Iodine, Molybdenum, Selenium, Iron, Zinc, Magnesium, and combinations thereof.

As noted above, compositions according to the present invention may include (6S)-5-methyl, 10-formylTHF and/or (6S) 5-formyl, 10-formylTHF or derivatives thereof. Derivatives include compounds formed from (6S)-5-methyl, 10-formylTHF and/or (6S) 5-formyl, 10-formylTHF which may be structurally distinct from (6S)-5-methyl, 10-formylTHF and/or (6S) 5-formyl, 10-formylTHF, but which retain the active function of folic acid. Non-limiting examples of such derivatives include salts of (6S)-5-methyl, 10-formylTHF and/or (6S) 5-formyl, 10-formylTHF, alkaline salts of (6S)-5-methyl, 10-formylTHF and/or (6S) 5-formyl, 10-formylTHF, esters of (6S)-5-methyl, 10-formylTHF and/or (6S) 5-formyl, 10-formylTHF, chelates of (6S)-5-methyl, 10-formylTHF and/or (6S) 5-formyl, 10-formylTHF and combinations thereof.

(6S)-5-methyl, 10-formylTHF and/or (6S) 5-formyl, 10-formylTHF or derivatives thereof may be present in an amount, for example, ranging from about 0.01 mg to about 50 mg. In certain embodiments, the compositions according to the present invention includes less than about 1000 μg, less than about 400 μg, less than about 300 μg, less than about 200 μg, or less than about 100 μg of (6S)-5-methyl, 10-formylTHF and/or (6S)-5-formyl, 10-formylTHF or derivatives thereof.

In one embodiment according to the present invention, the composition comprises (6S)-5-methyl, 10-formylTHF and/or (6S)-5-formyl, 10-formylTHF or derivatives thereof where the amount of (6S)-5-methyl, 10-formylTHF and/or (6S)-5-formyl, 10-formylTHF or derivatives thereof present is the daily recommended dietary allowance for a particular subject (e.g., woman of childbearing years). As will be appreciated, the daily recommended dietary allowance for particular nutrients is published by the National Academies Press (See National Academy of Sciences, Institute of Medicine, Food and Nutrition Board, “Dietary Reference Intakes (DRIs): Recommended Dietary Allowances and Adequate Intakes,” (2011), which is hereby incorporated by reference in its entirety).

In another embodiment, the amount of (6S)-5-methyl, 10-formylTHF and/or (6S)-5-formyl, 10-formylTHF in the composition is an amount that is less than the daily recommended dietary allowance of folate or derivatives thereof for a particular subject (e.g., woman of childbearing years, pregnant woman, or lactating woman).

In certain embodiments, the amount of (6S)-5-methyl, 10-formylTHF and/or (6S)-5-formyl, 10-formylTHF or derivatives thereof is at least about 25%, 30%, 35%, 40%, 45%, 50%, 55%, 60%, 65%, 70%, 75%, 80%, 85%, 90%, 95%, or 100% less than that recommended as a daily dietary allowance for a particular individual (e.g., a woman of childbearing age).

Effective doses of the compounds of the present invention vary depending upon many different factors, including type and stage of disease, mode of administration, target site, physiological state of the patient, other medications or therapies administered, and physical state of the patient relative to other medical complications. Treatment dosages need to be titrated to optimize safety and efficacy.

The compositions of the present invention can be administered in a single dose or multiple doses. The dosage can be determined by methods known in the art and can be dependent, for example, upon the individual's age, sensitivity, tolerance and overall well-being.

The dosage forms of the present invention may involve the administration of a composition according to the present invention in a single dose during a 24 hour period of time, a double dose during a 24 hour period of time, or more than two doses during a 24 hour period of time, or fractional doses to be taken during a 24 hour period of time. The double or multiple doses may be taken simultaneously or at different times during the 24 hour period.

It is also contemplated that the compositions according to the present invention are formulated for the dosage form to combine various forms of release, which include, without limitation, immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is performed using well known procedures and techniques available to the ordinary artisan.

Compositions described herein may contain compounds according to the present invention in an amount equivalent to that of the daily recommended dietary allowance of folate or derivatives thereof for a particular subject (e.g., woman of childbearing years). As will be appreciated, the daily recommended dietary allowance for particular nutrients is published by the National Academies Press (See National Academy of Sciences, Institute of Medicine, Food and Nutrition Board, “Dietary Reference Intakes (DRIs): Recommended Dietary Allowances and Adequate Intakes,” (2011), which is hereby incorporated by reference in its entirety). In another embodiment, the amount of the compounds according to the present invention in the composition is an amount that is less than the daily recommended dietary allowance of folate or derivatives thereof for a particular subject. In certain embodiments, the compounds according to the present invention are present in an amount that is at least about 5%, 10%, 15%, 20%, 25%, 30%, 35%, 40%, 45%, 50%, 55%, 60%, 65%, 70%, 75%, 80%, 85%, 90%, 95%, or 100% of that recommended as a daily dietary allowance of folate or derivatives thereof for a particular individual. In another embodiment, the compounds according to the present invention in the composition are present in an amount that is greater than the daily recommended dietary allowance of folate or derivatives thereof for a particular subject. In certain embodiments, the compounds according to the present invention are present in an amount that is at least about 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 450%, or 500% of that recommended as a daily dietary allowance of folate or derivatives thereof for a particular individual.

In one embodiment, the dose of compounds according to the present invention is in a range from about 0.5 mg/kg body weight per day to about 200 mg/kg body weight per day. In another embodiment, the dose is in a range from about 0.5 mg/kg body weight per day to about 150 mg/kg body weight per day. In another embodiment, the dose is in a range from about 0.5 mg/kg body weight per day to about 100 mg/kg body weight per day. In another embodiment, the dose is in a range from about 0.5 mg/kg body weight per day to about 95 mg/kg body weight per day. In another embodiment, the dose is in a range from about 0.5 mg/kg body weight per day to about 90 mg/kg body weight per day. In another embodiment, the dose is in a range from about 0.5 mg/kg body weight per day to about 85 mg/kg body weight per day. In another embodiment, the dose is in a range from about 0.5 mg/kg body weight per day to about 80 mg/kg body weight per day. In another embodiment, the dose is in a range from about 0.5 mg/kg body weight per day to about 75 mg/kg body weight per day. In another embodiment, the dose is in a range from about 0.5 mg/kg body weight per day to about 70 mg/kg body weight per day. In another embodiment, the dose is in a range from about 0.5 mg/kg body weight per day to about 65 mg/kg body weight per day. In another embodiment, the dose is in a range from about 0.5 mg/kg body weight per day to about 60 mg/kg body weight per day. In another embodiment, the dose is in a range from about 0.5 mg/kg body weight per day to about 55 mg/kg body weight per day. In another embodiment, the dose is in a range from about 0.5 mg/kg body weight per day to about 50 mg/kg body weight per day. In another embodiment, the dose is in a range from about 0.5 mg/kg body weight per day to about 45 mg/kg body weight per day. In another embodiment, the dose is in a range from about 0.5 mg/kg body weight per day to about 40 mg/kg body weight per day. In another embodiment, the dose is in a range from about 0.5 mg/kg body weight per day to about 35 mg/kg body weight per day. In another embodiment, the dose is in a range from about 0.5 mg/kg body weight per day to about 30 mg/kg body weight per day. In another embodiment, the dose is in a range from about 0.5 mg/kg body weight per day to about 25 mg/kg body weight per day. In another embodiment, the dose is in a range from about 0.5 mg/kg body weight per day to about 20 mg/kg body weight per day. In another embodiment, the dose is in a range from about 0.5 mg/kg body weight per day to about 15 mg/kg body weight per day. In another embodiment, the dose is in a range from about 0.5 mg/kg body weight per day to about 10 mg/kg body weight per day. In another embodiment, the dose is in a range from about 0.5 mg/kg body weight per day to about 5 mg/kg body weight per day. In another embodiment, the dose is in a range from about 0.5 mg/kg body weight per day to about 4 mg/kg body weight per day. In another embodiment, the dose is in a range from about 0.5 mg/kg body weight per day to about 3 mg/kg body weight per day. In another embodiment, the dose is in a range from about 0.5 mg/kg body weight per day to about 2 mg/kg body weight per day. In another embodiment, the dose is in a range from about 0.5 mg/kg body weight per day to about 1 mg/kg body weight per day.

In certain embodiments, the dose is or is at least about 0.1, 0.2, 0.3, 0.4, 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, or 200 mg/kg body weight per day.

In certain embodiments, the dose may be between about 10 mg/day and 15,000 mg/day, between about 100 mg/day and 2,500 mg/day, or between 250 mg to about 1,000 mg/day. In other embodiments, other ranges may be used, including, for example, 50-250 mg/day, 250-500 mg/day, and 500-750 mg/day. The amount of the compound required for prophylactic treatment, referred to as a prophylactically-effective dosage, is generally the same as described for effective dose.

In certain embodiments, the effective dose is between about 50 mg/day to about 150 mg/day. In certain embodiments, the dose is between about 10 mg/day and about 20 mg/day. In certain embodiments, the dose is between about 20 mg/day and about 30 mg/day. In certain embodiments, the dose is between about 30 mg/day and about 40 mg/day. In certain embodiments, the dose is between about 40 mg/day and about 50 mg/day. In certain embodiments, the dose is between about 50 mg/day and about 60 mg/day. In certain embodiments, the dose is between about 60 mg/day and about 70 mg/day. In certain embodiments, the dose is between about 70 mg/day and about 80 mg/day. In certain embodiments, the dose is between about 80 mg/day and about 90 mg/day. In certain embodiments, the dose is between about 90 mg/day and about 100 mg/day. In certain embodiments, the dose is between about 100 mg/day and about 110 mg/day. In certain embodiments, the dose is between about 110 mg/day and about 120 mg/day. In certain embodiments, the dose is between about 120 mg/day and about 130 mg/day. In certain embodiments, the dose is between about 130 mg/day and about 140 mg/day. In certain embodiments, the dose is between about 140 mg/day and about 150 mg/day. In certain embodiments, the dose is between about 150 mg/day and about 160 mg/day. In certain embodiments, the dose is between about 160 mg/day and about 170 mg/day. In certain embodiments, the dose is between about 170 mg/day and about 180 mg/day. In certain embodiments, the dose is between about 180 mg/day and about 190 mg/day. In certain embodiments, the dose is between about 190 mg/day and about 200 mg/day. In certain embodiments, the dose is between about 200 mg/day and about 250 mg/day. In certain embodiments, the dose is between about 250 mg/day and about 300 mg/day. In certain embodiments, the dose is between about 300 mg/day and about 350 mg/day. In certain embodiments, the dose is between about 350 mg/day and about 400 mg/day. In certain embodiments, the dose is between about 400 mg/day and about 450 mg/day. In certain embodiments, the dose is between about 450 mg/day and about 500 mg/day. In certain embodiments, the dose is between about 500 mg/day and about 550 mg/day. In certain embodiments, the dose is between about 550 mg/day and about 600 mg/day. In certain embodiments, the dose is between about 600 mg/day and about 650 mg/day. In certain embodiments, the dose is between about 650 mg/day and about 700 mg/day. In certain embodiments, the dose is between about 700 mg/day and about 750 mg/day. In certain embodiments, the dose is between about 750 mg/day and about 800 mg/day. In certain embodiments, the dose is between about 800 mg/day and about 850 mg/day. In certain embodiments, the dose is between about 850 mg/day and about 900 mg/day. In certain embodiments, the dose is between about 900 mg/day and about 950 mg/day. In certain embodiments, the dose is between about 950 mg/day and about 1000 mg/day. In certain embodiments, the dose is between about 1000 mg/day and about 1050 mg/day. In certain embodiments, the dose is between about 1050 mg/day and about 1100 mg/day. In certain embodiments, the dose is between about 1100 mg/day and about 1150 mg/day. In certain embodiments, the dose is between about 1150 mg/day and about 1200 mg/day. In certain embodiments, the dose is between about 1200 mg/day and about 1250 mg/day. In certain embodiments, the dose is between about 1250 mg/day and about 1300 mg/day. In certain embodiments, the dose is between about 1300 mg/day and about 1350 mg/day. In certain embodiments, the dose is between about 1350 mg/day and about 1400 mg/day. In certain embodiments, the dose is between about 1400 mg/day and about 1450 mg/day. In certain embodiments, the dose is between about 1450 mg/day and about 1500 mg/day.

An effective dose or amount, and any possible effects on the timing of administration of the formulation, may need to be identified for any particular composition of the present invention. This may be accomplished by routine experiment, using one or more groups of animals, or in human trials if appropriate. The effectiveness of any subject composition and method of treating may be assessed by administering the composition and assessing the effect of the administration by measuring one or more applicable indices, and comparing the post-treatment values of these indices to the values of the same indices prior to treatment.

Folate deficiencies can be caused by inadequate dietary intake, or can be disease-induced. Patients with disease-induced folate deficiency, which can be specific to organs and/or cell types, can require folate intakes above the Recommended Dietary Allowance and that cannot be met through a food-based diet. These patients require pro-vitamin forms of folate to meet their distinct nutritional needs.

Accordingly, another aspect of the present invention relates to a method of increasing levels of bioactive (6S)-THF in a subject. This method includes administering to the subject an amount of one or more compounds of the present invention effective to increase levels of (6S)-THF levels in the subject.

In one embodiment, cerebrospinal fluid (CSF) levels of THF and its one-carbon forms are increased. Suitable compositions, dosages, formulations, modes of administration, etc., are described above.

The subject of methods of the present invention may be a mammal. The subject may be a human. The subject may be a laboratory animal (e.g., a rodent, canine, or simian). In another embodiment, the subject is a male. In another embodiment, the subject is a female. In another embodiment, the subject is a child. In another embodiment, the subject may also be a child. The child may be younger than 6, 5, 4, 3, or 2 years old. The subject may also be an infant (0-1 year).

The subject may be a healthy individual. The subject may be an individual at risk for developing (or be suffering from) a folate deficiency. The subject may be an individual at risk for developing (or be suffering from) a disease, disorder, or condition associated with folate deficiency. The subject may also be one at risk for developing (or be suffering from) a folate-responsive disease, disorder, or condition. Also contemplated are subjects having symptoms of any of the above deficiencies, diseases, disorder, or conditions.

By mere way of example, compounds and/or compositions according to the present invention may be administered to subjects having or at risk for developing: neurodegenerative diseases or disorders; cerebral folate deficiency (CFD) and related symptoms; a fetus with folate-responsive disorder (e.g., a neural tube defect, a cleft lip defect, and/or a cleft palate defect); cancer; stroke; and/or megaloblastic anemia due to folate deficiency.

In certain embodiments, the compounds according to the present invention are administered under conditions effective to treat the deficiency, disease, disorder, or condition described herein. As used herein, treating refers to a reduction in disease state or condition as compared to the disease state or condition without or before administering the compound or composition according to the present invention. Such treatment can be, but is not limited to, the complete ablation of the disease, condition, or the symptoms of the disease or condition. For example, a disclosed method is considered to be a treatment if there is a reduction in one or more symptoms of the disease (e.g., tumor size) in a subject with the disease when compared to native levels in the same subject or control subjects. Thus, the reduction can be a 5, 10, 20, 30, 40, 50, 60, 70, 80, 90, 100%, or any amount of reduction in between as compared to native or control levels. It is also understood and contemplated herein that treatment can refer to any reduction in the progression of a disease or cancer. Thus, for example, methods of reducing the effects of a cancer is considered to be a treatment if there is a reduction in the tumor growth rate relative to a control subject or tumor growth rates in the same subject prior to the treatment. It is understood that the reduction can be a 5, 10, 20, 30, 40, 50, 60, 70, 80, 90, 100%, or any amount of reduction in between as compared to native or control levels.

In one embodiment, the subject is one that has or is at risk for developing a neurodegenerative or brain disorder.

In one embodiment, the subject is one that has or is at risk for developing cerebral folate deficiency (CFD). CFD is defined by low cerebrospinal fluid (CSF) folates. The predominant and biologically active folate form 5-methylTHF (5MTHF) is the single folate form known to pass the blood-CSF barriers and is significantly reduced in the CSF. CFD may be characterized as any neurological syndrome associated with low CSF 5MTHF, the active folate metabolite, in the presence of normal folate metabolism outside the nervous system.

CFD is a collective name for any neurological or neuropsychiatric syndrome associated with low CSF 5MTHF. The term “CFD” is meant to include any disease associated with CFD (also referred to as a CFD-related disease) and any secondary form of CFD. CFD can result either from a disturbed folate transport or from an increased folate turnover (utilization and/or breakdown) in the central nervous system (CNS). CFD is a highly debilitating disease. The estimated incidence for CFD is 1:1000 to 1:1500. The real incidence is probably much higher. CFD can affect people of all ages with a variable clinical phenotype including slowly progressive neurologic dysfunction affecting infants, children, and even adults.

Infantile-onset CFD is a neurological syndrome that develops about 4-6 months after birth and may be characterized as any neuro-psychiatric condition associated with low spinal fluid (CSF) 5MTHF but normal folate status outside the CNS.

Secondary CFD is known to develop during chronic use of antifolate agents like methotrexate, anticonvulsant drugs, and carbidopa, and in various known conditions such as Rett syndrome, Aicardi-Goutières syndrome, 3-phosphoglycerate dehydrogenase deficiency, dihydropteridine reductase deficiency, aromatic amino acid decarboxylase deficiency, mitochondrial encephalopathies and Kearns-Sayre syndrome.

In one embodiment, the subject is a pregnant woman, lactating woman, or woman of childbearing age. In one embodiment of the present invention, administering one or more compounds according to the present invention to the subject prevents development of, or reduces the risk for developing, folate-deficiency related birth defects. One class of folate-deficiency related birth defects is neural tube defects. Neural tube defects include, but are not limited to, anencephaly, encephaloceles, hydranencephaly, iniencephaly, schizencephaly, spina bifida, lipomyelomeningocele, lipomeningocele, and tethered cord.

In one embodiment, the woman is a woman of childbearing age that is attempting to become pregnant. A woman of childbearing age that is attempting to become pregnant includes, for example, women using assisted reproductive technology (e.g., in vitro fertilization (IVF)), women monitoring fertility, and women generally attempting to conceive a child. In one embodiment, the compound or composition according to the present invention is administered in conjunction with a fertility agent (e.g., FSH, GnRh, Clomiphene citrate, or HMG). In another embodiment, the subject is a woman who is at risk for developing a fetus with folate-deficiency related birth defects. This includes, for example, a woman who is predisposed to for developing a fetus with folate-deficiency related birth defects. For example, a woman may be at risk for developing a fetus with folate-deficiency related birth defects due to low consumption of folate, obesity, and/or diabetes. This also includes, for example, a woman who has developed a fetus with folate-deficiency related birth defects in the past.

In one embodiment, the subject is at risk for experiencing a stroke. In another embodiment, the subject has hypertension.

In one embodiment, the subject is one that has or is at risk for developing folate-deficiency related megaloblastic anemia.

In one embodiment, the subject is one that has or is at risk for developing cancer. The cancer may be, e.g., intestinal cancer. Those at risk for developing intestinal cancer include, for example, subjects having one or more risk factors for developing intestinal cancer. Risk factors for developing intestinal cancers include, for example, a personal history of colorectal cancer or polyps; inflammatory intestinal conditions (e.g., Chronic inflammatory diseases of the colon, such as ulcerative colitis and Crohn's disease); inherited syndromes that increase cancer risk (genetic syndromes such as familial adenomatous polyposis and hereditary nonpolyposis colorectal cancer); family history of intestinal cancers (e.g., colon cancer and colon polyps); diabetes; obesity; smoking; heavy use of alcohol; and radiation therapy for cancer.

Compounds according to the present invention can be administered alone or in conjunction with other or second therapies.

In one embodiment, the second therapy is a stroke therapy or stroke preventive therapy. In one embodiment, the therapy is enalapril.

In another embodiment, the second therapy is a cancer therapy (e.g., chemotherapeutic agents, radiation, surgery, or combinations thereof). In one embodiment, the cancer therapy is a chemotherapeutic. In one embodiment, the chemotherapeutic is selected from the group consisting of alkylating agents, antimetabolites, anthracyclines, antitumor antibiotics, platinum-based chemotherapeutics, and plant alkaloids.

Particular examples of chemotherapeutics or chemotherapy include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, magastrol, melphalan, mercaptopurine, oxaloplatin, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, taxol, and combinations thereof.

In one embodiment, the cancer therapy is radiation. The source of radiation can be either external or internal to the patient being treated. When the source is external to the patient, the therapy is known as external beam radiation therapy (EBRT). When the source of radiation is internal to the patient, the treatment is called brachytherapy (BT).

In another embodiment, the cancer therapy is surgery.

In one embodiment, the compound according to the present invention and the cancer therapy are administered simultaneously. In another embodiment, the compound according to the present invention is administered prior to the cancer therapy. In yet another embodiment, the compound according to the present invention is administered following the cancer therapy.

Compounds according to the present invention can also be conjugated to other therapeutic molecules through, e.g., a carboxylic group of the compounds. Such conjugation is known in the art, e.g., U.S. Patent Application Publication No. 2002/0077279, which is hereby incorporated by reference in its entirety.

Compounds of the present invention can be used as an over-the-counter dietary supplement for healthy individuals that is more effective than folic acid in meeting dietary folate needs.

Compounds of the present invention can be used as a nutritional supplement for those at increased risk for cancer.

Compounds of the present invention can be used as a medical treatment at high doses to address whole-body and/or tissue-specific folate deficiencies, including those induced or associated with brain disease.

Compounds of the present invention can be administered as an adjuvant to anticancer chemotherapy, metformin use, or other pharmaceutical treatment for disease prevention, treatment or management.

Compounds of the present invention can be used in laboratory based experiments as a source of stable reduced folates for in vitro, cell culture, and animal studies.

Kits (e.g., fertility kits) including compounds and compositions according to the present invention are also contemplated. In one embodiment, the kit includes one or more doses of one or more compounds according to the present invention or a composition comprising one or more compounds according to the present invention, an ovulation monitoring component, and a set of instructions for timing and utilization of the compounds and/or compositions according to the present invention together with timing intercourse to maximize fertility potential. The monitoring component may include, for example, a monitor of salivary electrolytes, basal body temperature, or luteinizing hormone (LH), to predict and determine when ovulation will/has occurred. In one embodiment, the ovulation monitoring component includes a basal body temperature chart for cataloging the typical menstrual cycle for the female.

Although the present invention has been described for the purpose of illustration, it is understood that such detail is solely for that purpose and variations can be made by those skilled in the art without departing from the spirit and scope of the invention which is defined by the following claims.

The following statements are potential claims that may be converted to claims in a future application. No modifications of the following statements should be allowed to affect the interpretation of claims which may be drafted when this provisional application is converted into a regular utility application.

### EXAMPLES

The following examples are provided to illustrate embodiments of the present invention but are by no means intended to limit its scope.

### Example 1—Synthesis of 5-Formyl, 10-formyl-tertrahydrofolate

(6S) 5-Formyltetrahydrofolate (100 mg) was dissolved in neat formic acid (5 ml). The mixture was incubated at room temperature under nitrogen for 60 minutes to allow formation of 5,10-methenyltetrahydrofolate. Then, 2-mercaptoethanol (200 μL) was added to the reaction mixture. Solid sodium bicarbonate was added until the pH reached 5.5. 1-Ethyl-3-(3-Dimethylaminopropyl)carbodiimide (EDAC) (1 g) was then added and nitrogen was bubbled through the mixture for 5 hours. The mixture was diluted 2× with water and extracted 3 times with ethylacetate. The organic layer was discarded. CaCl₂ (100 mg) was added to this solution. 5-Formyl, 10-formyl-tertrahydrofolate was precipitated with EtOH (5×), pellet was washed 2 times with MeOH. The compound was dried by using a speedvac.

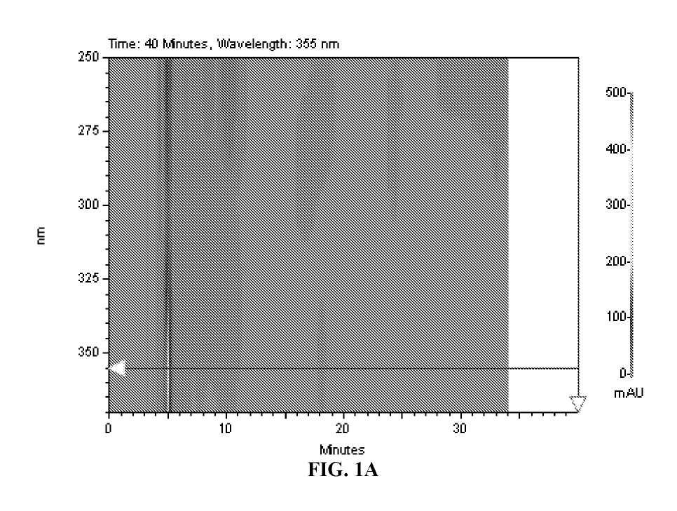

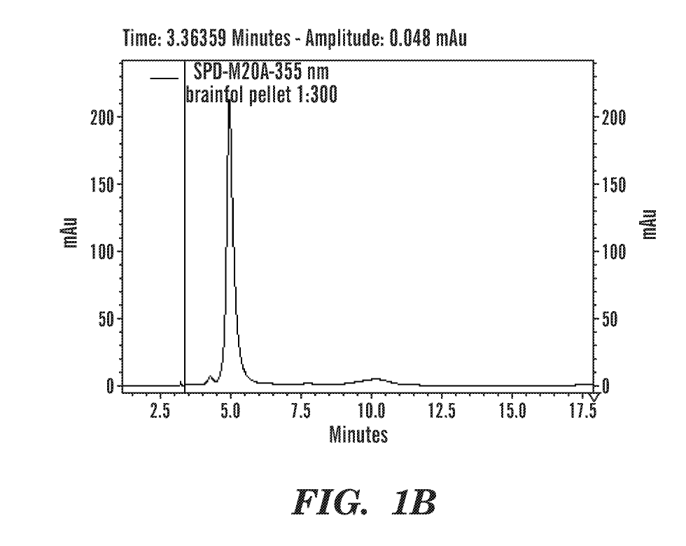

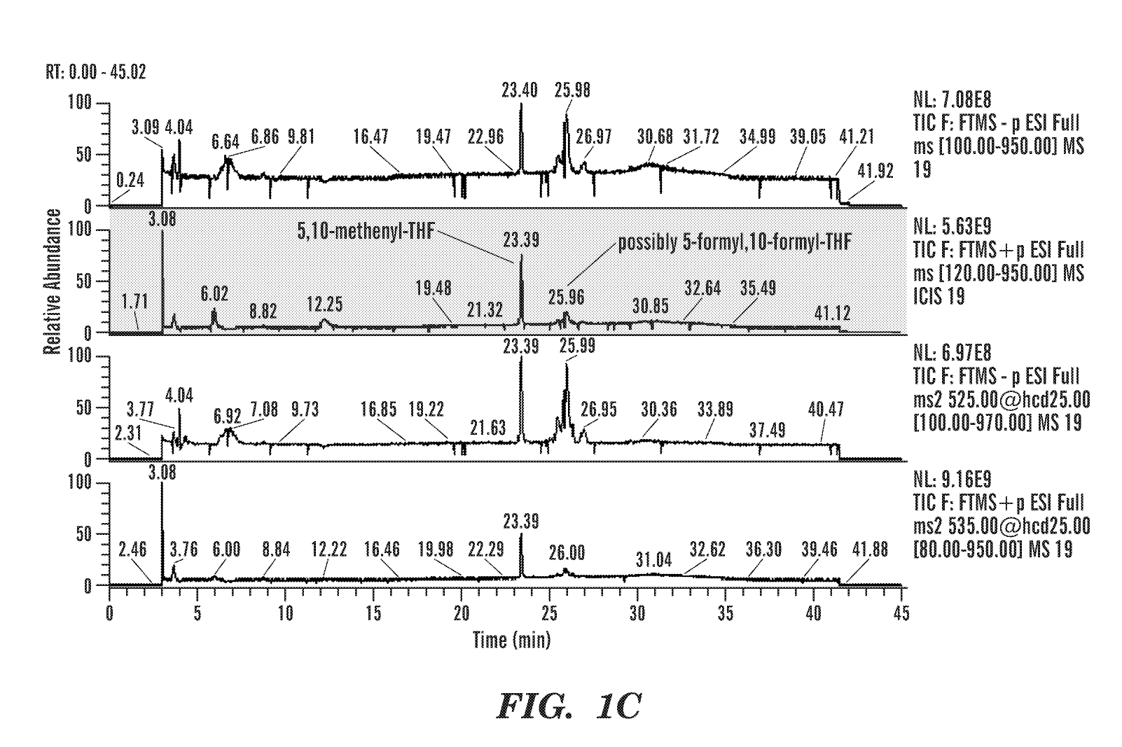

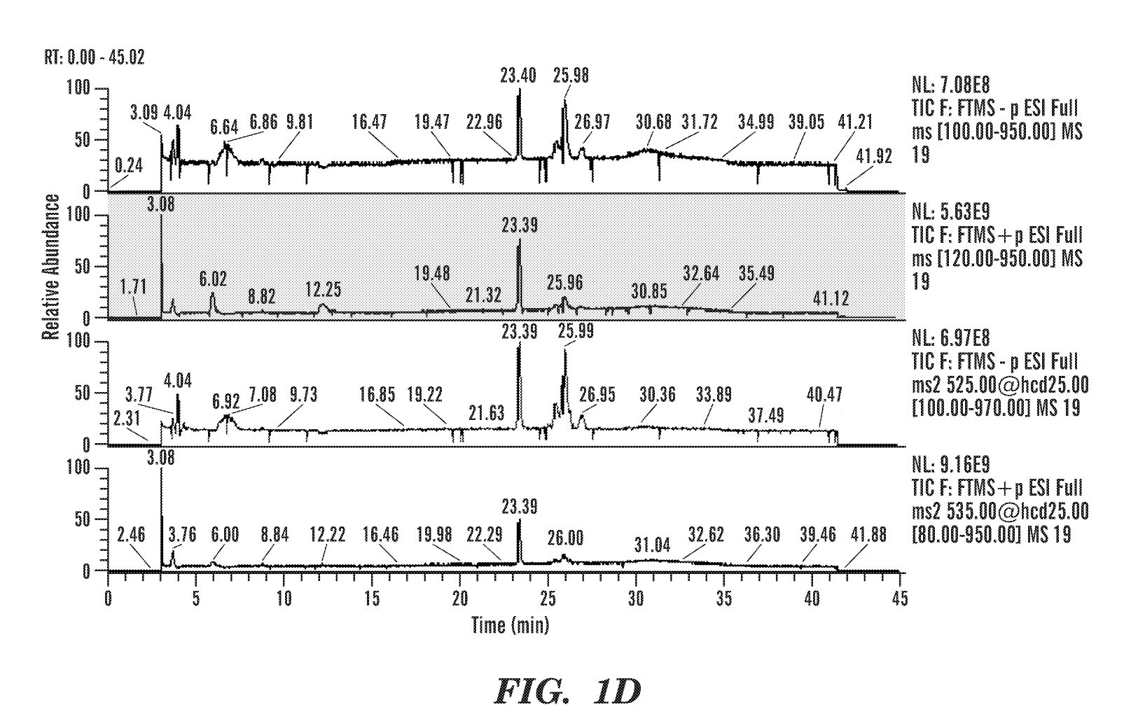

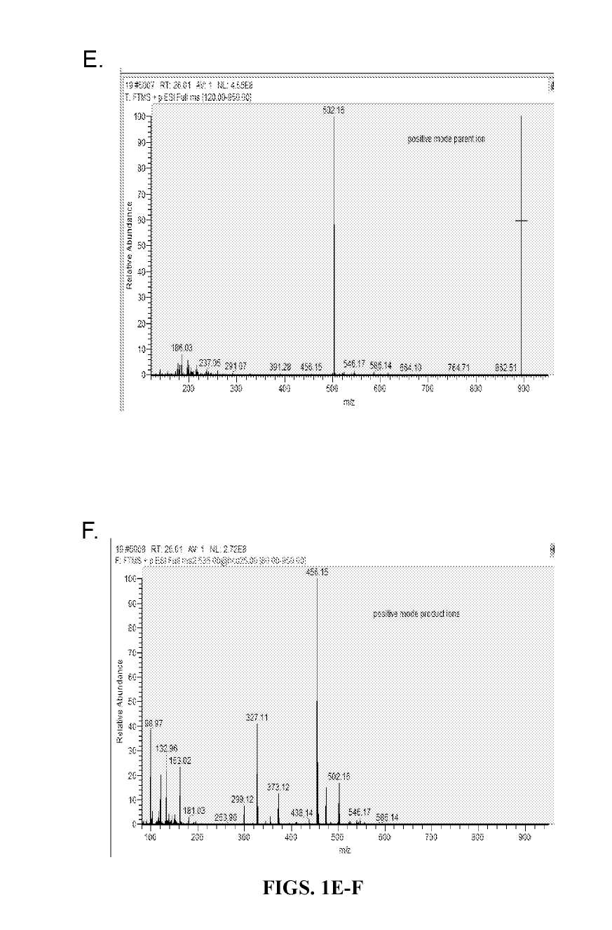

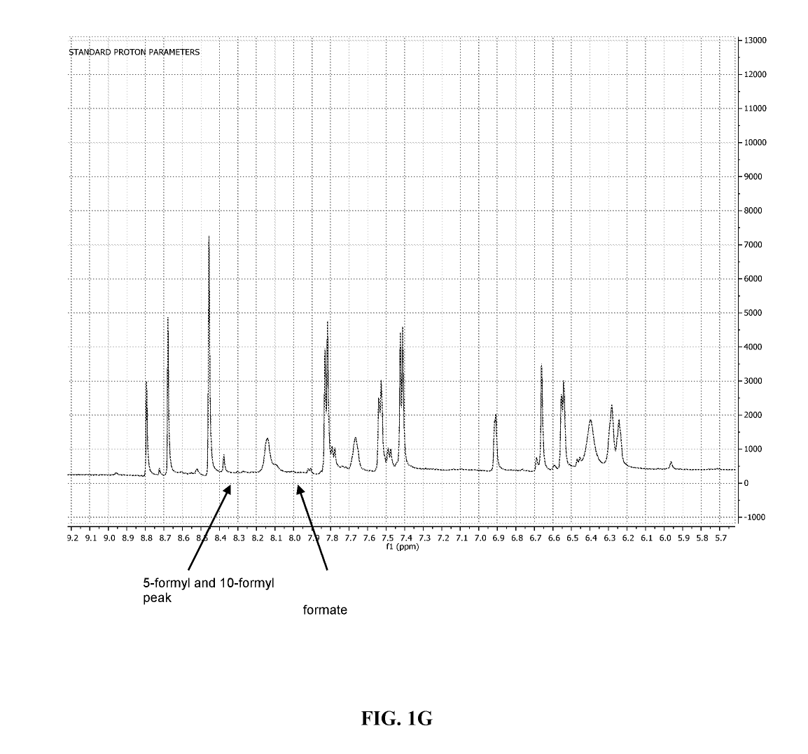

HPLC, NMR, and mass spectrometry spectra of (6)-5-Formyl, 10-formyl-tertrahydrofolate are shown in FIGS. 1A-G. NMR and mass-spectrometry spectra was taken from unpurified reaction.

### Example 2—Synthesis of 5-Methyl, 10-formyltetrahydrofolate

(6S)-5-Methyl-tetrahydrofolate (100 mg) was dissolved in of 98% formic acid (1 ml). The reaction mixture was heated at 110° C. for 40 minutes. Solution was dried under a stream of nitrogen. Pellet was dissolved the in 1M ammonium bicarbonate (2 ml). The aqueous solution was extracted 3 times with an equal volume of ethyl acetate. The organic layer was discarded. Crude (6S)-5-methyl, 10-formyl-tetrahydrofolate was precipitated with 5 volumes of methanol. Pellet was washed 5 times with methanol and dried under vacuum.

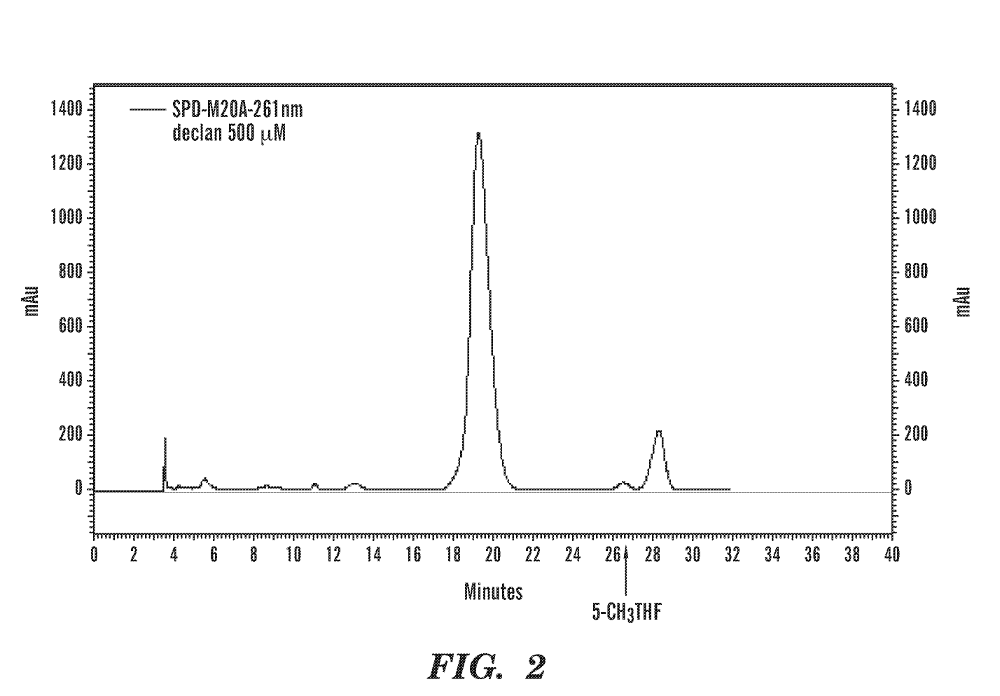

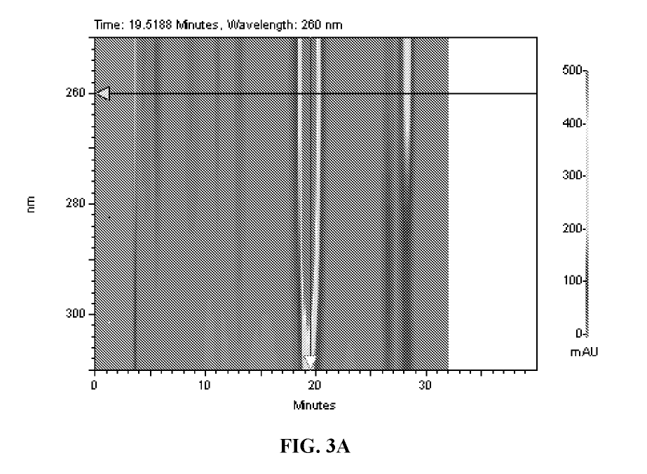

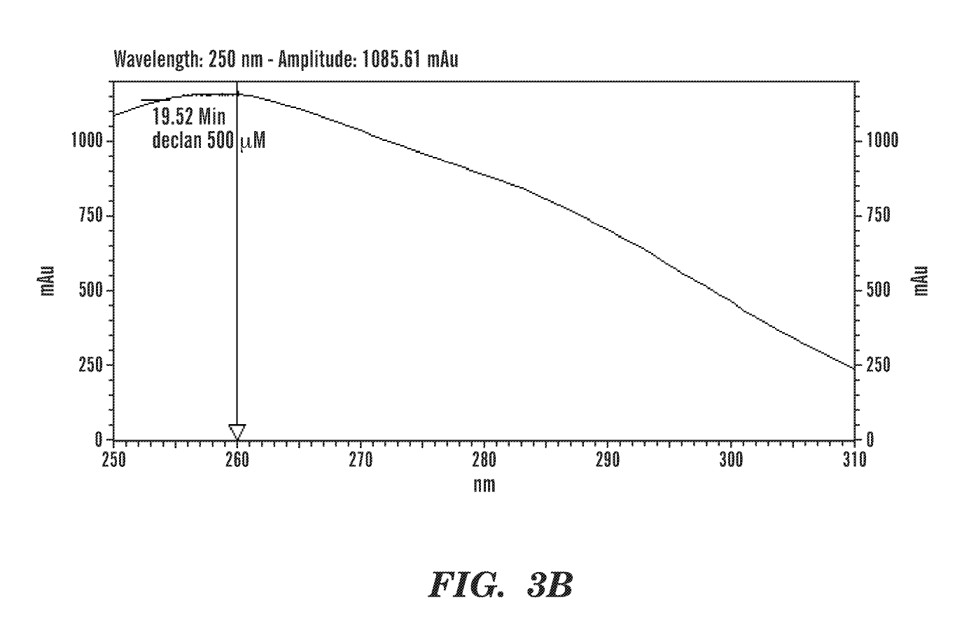

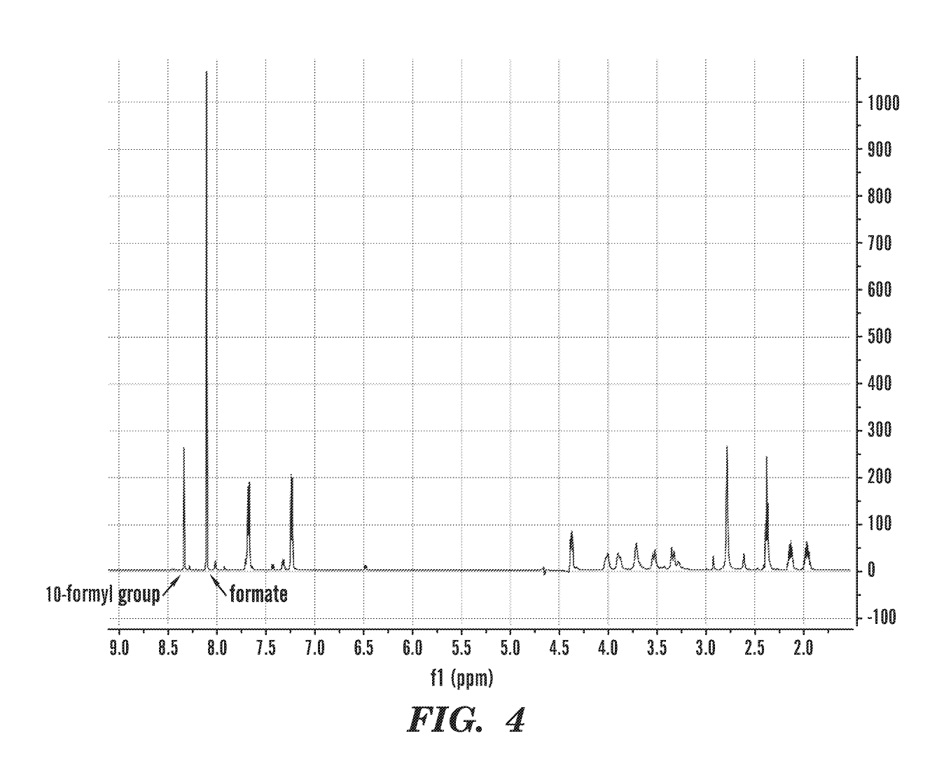

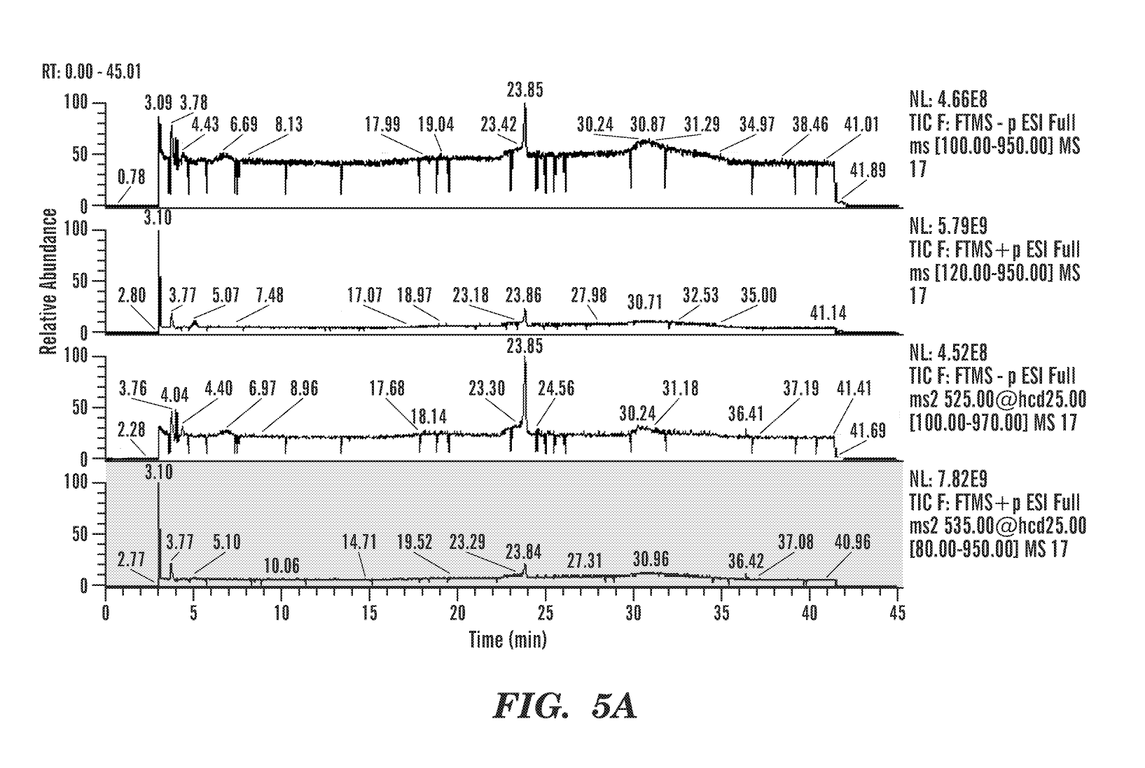

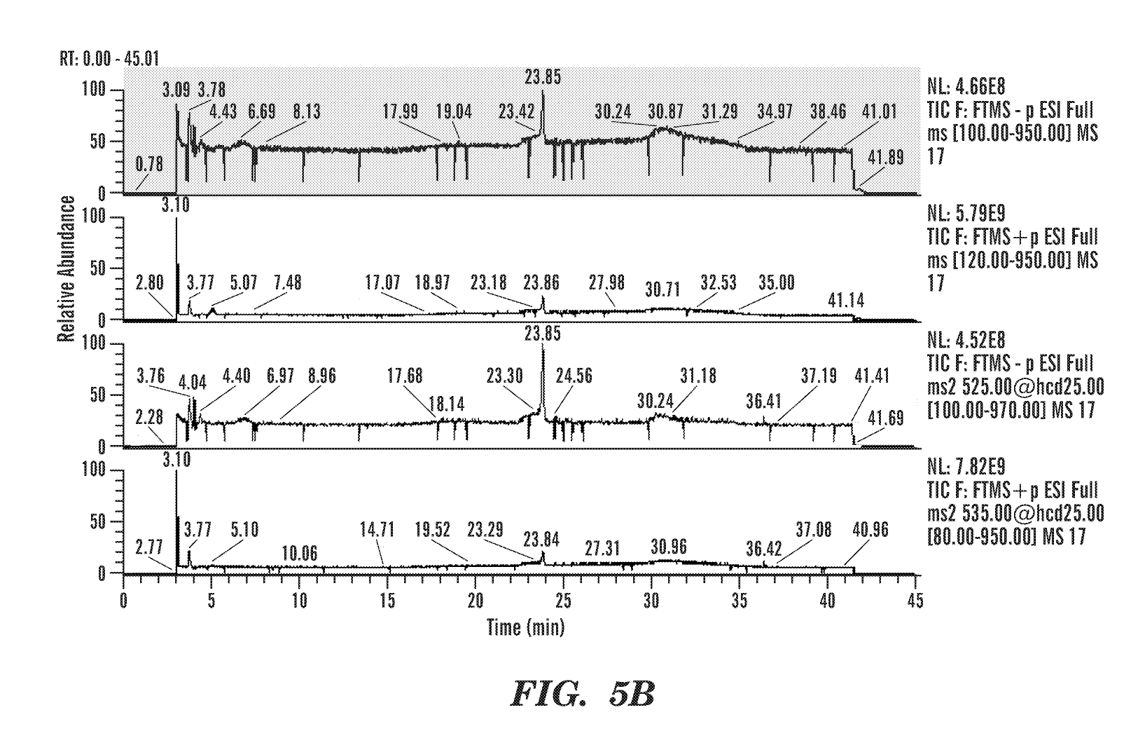

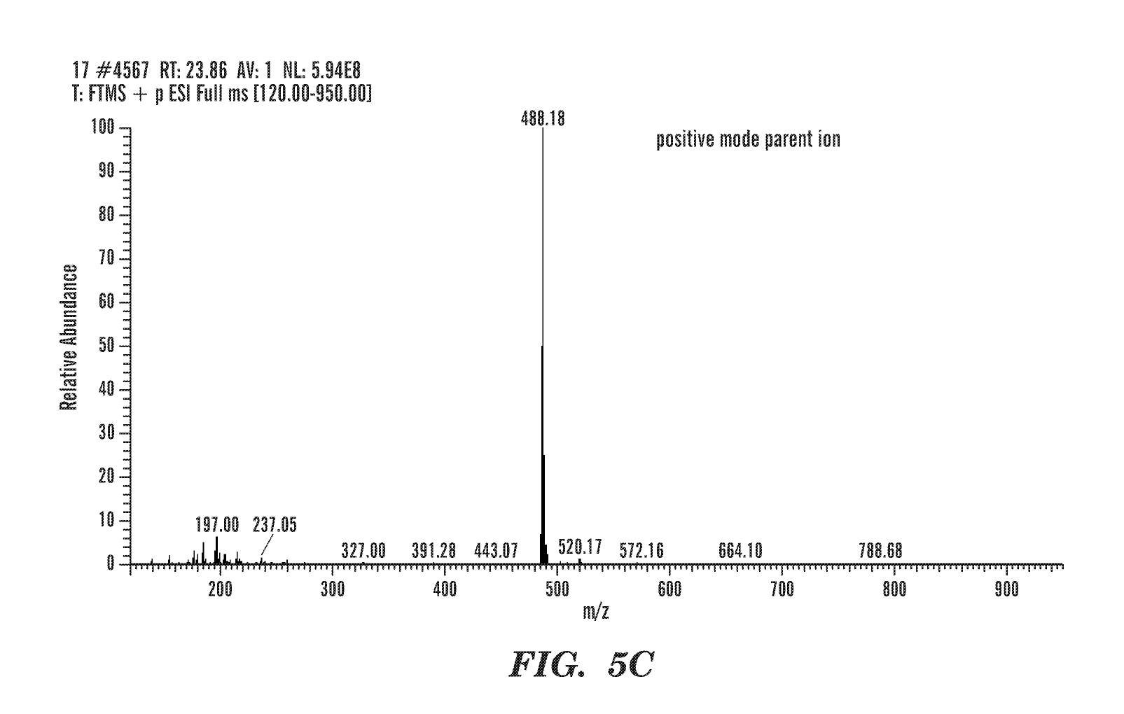

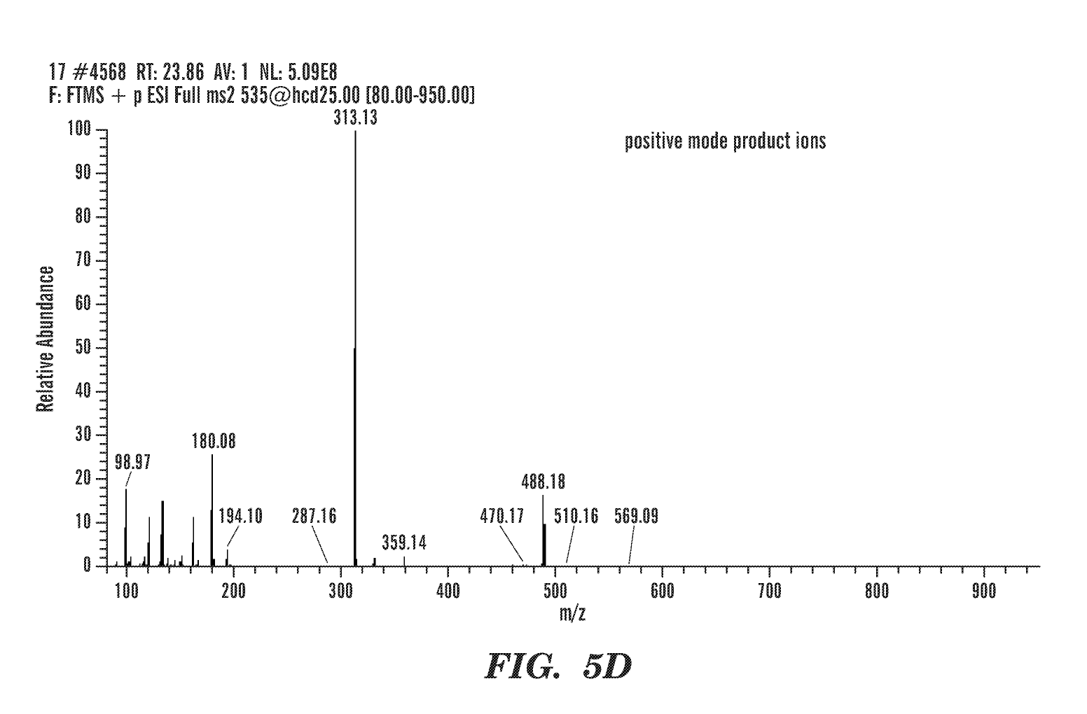

HPLC trace of 5-methyl, 10-formyl-tertrahydrofolate indicated greater than 90% purity (FIG. 2). Chromatogram was taken at 260 nm wavelength. UV, NMR and mass spectrometry spectra of (6)-5-methyl, 10-formyl-tetrahydrofolate are shown in FIGS. 3A-B, FIG. 4, and FIGS. 5A-D.

### Example 3—Methods

### Effect of (6)-5-Methyl, 10-formyltetrahydrofolate and (6)-5-Formyl, 10-formyltetrahydrofolate on Cell Proliferation

HeLa cells and immortalized mouse fibroblasts (CHIP) cells were plated in 96-well plates, 1000 cells per well. Total cell number was monitored over time using bright field image analysis on a Celigo cytometer (Nexcelom, Inc.) according to manufacturers' protocols. Data was represented as average±standard deviation for 10 biological replicates per condition. Cells were cultured in Defined αMEM culture media (lacking folate, serine, glycine, methionine, nucleosides/nucleotides, and pyridoxine) which was supplemented with 200 μM methionine, 0.5 g/L pyridoxine, and varying concentrations of folic acid, (6S)-Leucovorin or novel folate derivative. Before plating for proliferation assays, HeLa cells and CHIP cells were cultured for three doublings in Defined αMEM lacking folate to deplete intracellular folates.

Total Folate Measurement

HeLa cells were cultured in Defined αMEM (as described above) lacking folate for two doublings to deplete cellular folates, then plated into 6-well plates (1000 cells/well) and cultured for two cell doublings with Defined αMEM culture media (described above) supplemented with 200 μM methionine, 0.5 g/L pyridoxine, and either 2 nM or 20 nM (6S)-Leucovorin or novel folate derivative. Total intracellular folates were quantified using a microbiological (Lactobacillus casei, or L. casei) assay as previously described (Suh et al., “Purification and Properties of a Folate-Catabolizing Enzyme,” J. Biol. Chem. 275(45):35646-35655 (2000), which is hereby incorporated by reference in its entirety). Data is represented as mean±SEM of three biological replicates per condition.

Chemical Stability of Synthetic Reduced Folates

Folic acid and various reduced folates were incubated in Defined αMEM (described above) in sterile Eppendorf tubes at 65° C. and sampled at 0, 0.5, 1, 2, 4, 8, and 24 hours. The sampled aliquots were immediately mixed with L. casei extraction buffer containing sodium ascorbate to protect folates from oxidative degradation and frozen at −80° C. The L. casei assay was performed as previously described (Suh et al., “Purification and Properties of a Folate-Catabolizing Enzyme,” J Biol. Chem. 275(45):35646-35655 (2000), which is hereby incorporated by reference in its entirety).

Folic acid and (6S)-5-formyl, 10-formyl-tetrahydrofolate were shipped to Dyets, Bethlehem, Pa. as folate sources for the formulation of AIN-93G, a defined growth diet formulated for rodents by the American Institute of Nutrition. The stability of folic acid and (6S)-5-formyl, 10-formyl-tetrahydrofolate in food pellets was determined over a 10 week period.

### Example 4—Results of Examples 1-3

Folate Accumulation and Proliferation in Mammalian Cell Lines

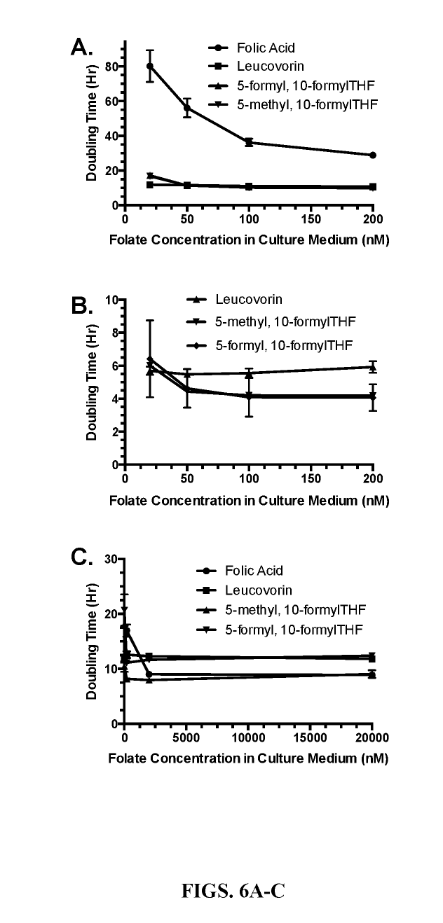

Proliferation of HeLa cells and CHIP cells cultured over a range of concentrations of (6S)-5-methyl, 10-formyltetrahydrofolate, (6S)-5-formyl, 10-formyltetrahydrofolate, (6S)-Leucovorin, and folic acid was quantified using bright-field identification of cells cultured for approximately three doublings (FIGS. 6A-C). (6S)-5-Methyl, 10-formyltetrahydrofolate and (6S)-5-formyl, 10-formyltetrahydrofolate were similar at supporting cell growth compared to Leucovorin and far superior to folic acid. Leucovorin is a reduced form of folate, and is unstable in acid, because it converts to 5,10-methenyltetrahydrofolate and should not be taken orally, whereas the novel folate derivatives are acid stable and cannot convert to 5,10-methenyltetrahydrofolate.

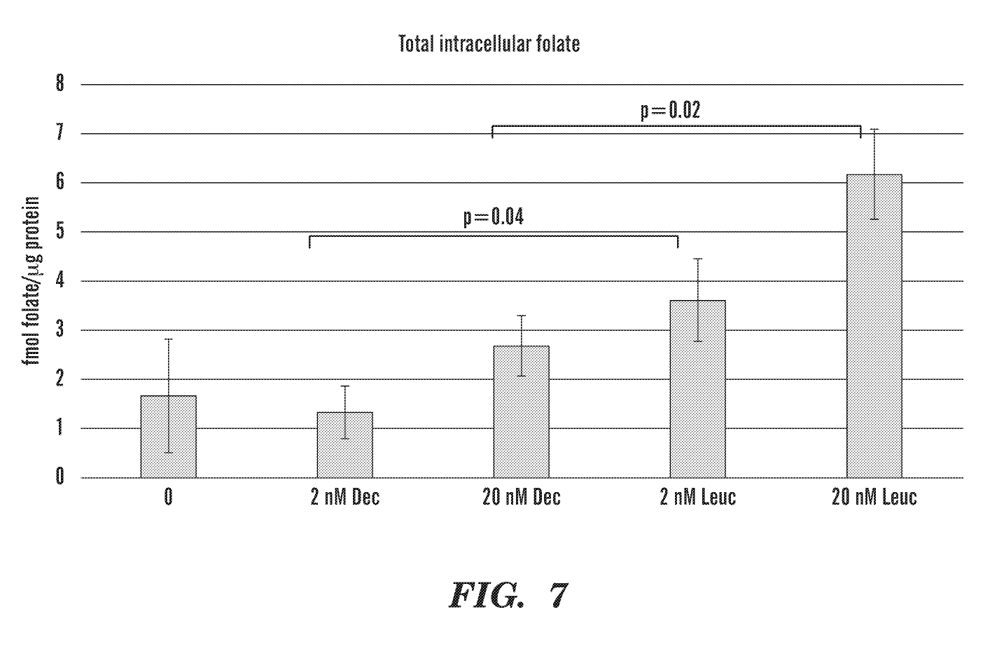

When HeLa cells were cultured under similar conditions and intracellular total folate levels quantified, total intracellular folate levels were higher with (6S)-Leucovorin than with (6S)-5-methyl, 10-formyltetrahydrofolate (FIG. 7), indicating that either (6S) 5-methyl, 10-formyltetrahydrofolate is not transported as effectively as (6S)-Leucovorin or that (6S)-5-methyl, 10-formyltetrahydrofolate is not as efficiently converted to the natural folate cofactor. Interestingly, total cellular folate in HeLa cells cultured with 20 nM (6S) 5-methyl, 10-formyltetrahydrofolate is equivalent to total cellular folate in HeLa cells cultured in 2 nM (6S)-Leucovorin, as suggested by identical growth curves (FIGS. 6A-C).

### Chemical Stability of (6S)-5-Methyl, 10-formyltetrahydrofolate and (6S)-5-Formyl, 10-formyltetrahydrofolate

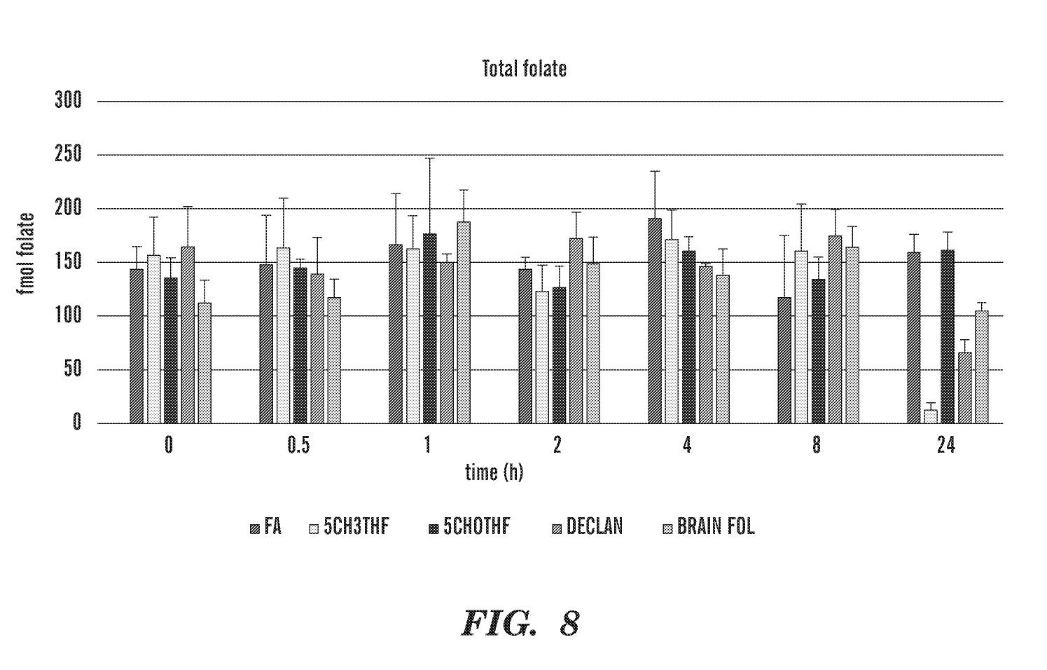

All folates supported growth in the microbiological assay after 8 hours incubation at 65° C., pH 7.2 (FIG. 8). This indicated that after 8 hours incubation all folates were chemically intact. After 24 hours, however, 5-methyl-tetrahydrofolate was completely destroyed (unable to support growth), whereas folic acid and (6S)-Leucovorin continued to support growth in this assay. Both (6S)-5-methyl, 10-formyltetrahydrofolate and (6S)-5-formyl, 10-formyltetrahydrofolate under these conditions were chemically more stable than 5-methyltetrahydrofolate (FIG. 8).

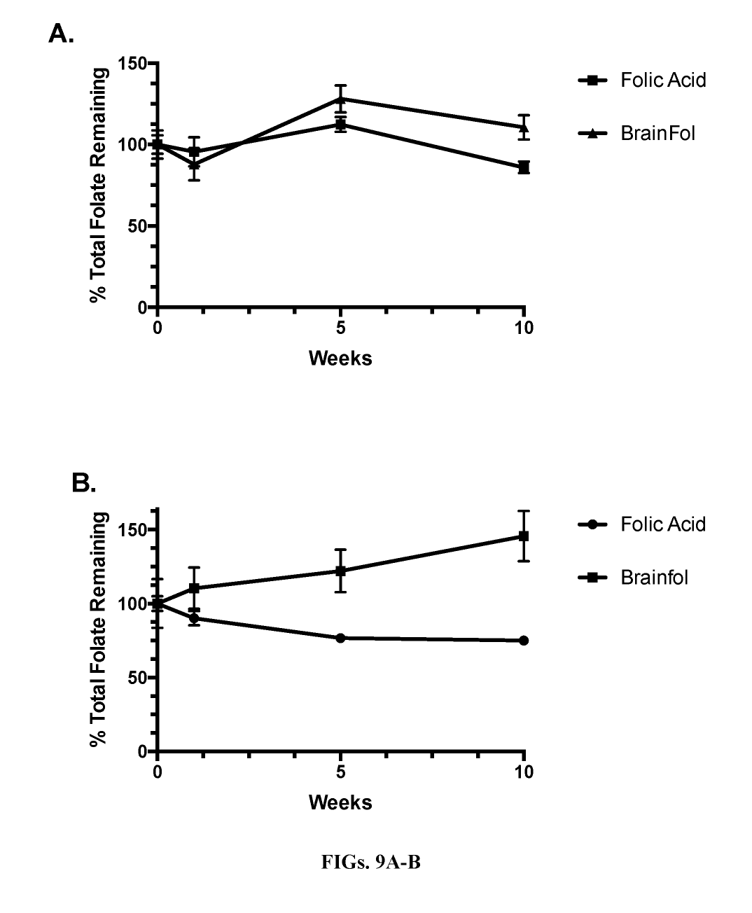

Folic acid and (6S)-5-formyl, 10-formyltetrahydrofolate are completely stable as a component of AIN-93G rodent food for at least 10 weeks, and both promote mouse growth over at least an 8-week period (FIGS. 9A-B).

Although the invention has been described in detail, for the purpose of illustration, it is understood that such detail is for that purpose and variations can be made therein by those skilled in the art without departing from the spirit and scope of the invention which is defined by the following claims.

1. A compound of Formula I: 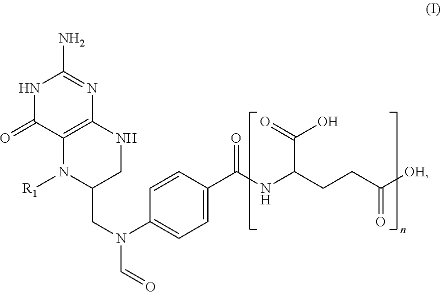
wherein
R₁ is a C₁-C₆ alkyl or 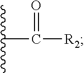
R₂ is H or C₁-C₆ alkyl; and
n is 1 to 8,
or a pharmaceutically acceptable salt thereof, or a solvate thereof.

2. The compound according to claim 1, wherein the compound is (6S) 5-methyl, 10-formyltetrahydrofolate having the formula:

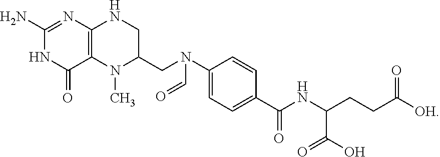 [SMILES: CN1c2c(nc(N)[nH]c2=O)NCC1CN(C=O)c1ccc(C(=O)NC(CCC(=O)O)C(=O)O)cc1]

3. The compound according to claim 1, wherein the compound is (6S)-5-formyl, 10-formyltetrahydrofolate having the formula:

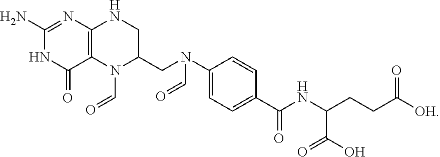 [SMILES: Nc1nc2c(c(=O)[nH]1)N(C=O)C(CN(C=O)c1ccc(C(=O)NC(CCC(=O)O)C(=O)O)cc1)CN2]

4. A pharmaceutical or dietary composition comprising:
the compound according to claim 1 and a pharmaceutically or dietetically suitable carrier.

5. The pharmaceutical or dietary composition according to claim 4, wherein the composition is part of a nutraceutical composition, pharmaceutical composition, functional food, functional nutrition product, medical food, medical nutrition product, or dietary supplement.

6. The pharmaceutical or dietary composition according to claim 4 further comprising:
one or more vitamins, minerals, trace elements, or combinations thereof.

7. The pharmaceutical or dietary composition according to claim 6, wherein the one or more vitamins, minerals, trace elements, or combinations thereof is selected from the group consisting of Vitamin A, Vitamin C, Vitamin D, Vitamin E, Vitamin K, Thiamin, Riboflavin, Niacin, Vitamin B12, Vitamin B6, Pantothenic Acid, Biotin, Choline, Chromium, Copper, Iodine, Molybdenum, Selenium, Iron, Zinc, Magnesium, and combinations thereof.

8. The pharmaceutical or dietary composition according to claim 4, wherein the compound is selected from (6S)-5-methyl, 10-formylTHF; (6S)-5-formyl, 10-formylTHF; derivatives thereof; and combinations thereof present in an amount that is less than about 1000 μg, less than about 400 μg, less than about 300 μg, less than about 200 μg, or less than about 100 μg.

9. The pharmaceutical or dietary composition according to claim 4, wherein the compound is selected from (6S)-5-methyl, 10-formylTHF; (6S)-5-formyl, 10-formylTHF; derivatives thereof; and combinations thereof in an amount at least about 50% less than that recommended as a daily dietary allowance for a woman of childbearing age.

10. The pharmaceutical or dietary composition according to claim 4, wherein the composition is in capsule, tablet, granule, or lozenge form.

11. A method of supplementing the dietary needs of a subject, said method comprising:
administering to the subject a dietary supplementing effective amount of the compound according to claim 1.

12. A method of increasing (6S)-tetrahydrofolate levels in a subject, the method comprising:
selecting a subject; and
administering to the selected subject an effective amount of the compound according to claim 1 to increase (6S) tetrahydrofolate levels in the selected subject.

13. The method according to claim 11, wherein the subject is a human.

14. The method according to claim 11, wherein the subject has or is at risk for developing folate deficiency.

15. The method according to claim 11, wherein the subject has or is at risk for developing a folate-responsive disease, disorder, or condition.

16. The method according to claim 14, wherein the subject has or is at risk for developing a neurodegenerative or brain disorder; cerebral folate deficiency (CFD); stroke; hypertension; megaloblastic anemia; or cancer.

17. The method according to claim 11, wherein the subject is a pregnant woman, lactating woman, or woman of childbearing age.

18. The method according to claim 17, wherein the woman is a woman of childbearing age that is attempting to become pregnant.

19. The method according to claim 11, wherein the compound or composition is administered as part of a nutraceutical composition, pharmaceutical composition, functional food, functional nutrition product, medical food, medical nutrition product, or dietary supplement.

20. The method according to claim 11, wherein said administering further comprises:
administering one or more vitamins, minerals, trace elements, or combinations thereof.

21. The method according to claim 20, wherein the one or more vitamins, minerals, trace elements, or combinations thereof is selected from the group consisting of Vitamin A, Vitamin C, Vitamin D, Vitamin E, Vitamin K, Thiamin, Riboflavin, Niacin, Vitamin B12, Vitamin B6, Pantothenic Acid, Biotin, Choline, Chromium, Copper, Iodine, Molybdenum, Selenium, Iron, Zinc, Magnesium, and combinations thereof.

22. The method according to claim 11, wherein the compound is selected from (6S)-5-methyl, 10-formylTHF; (6S)-5-formyl, 10-formylTHF; derivatives thereof; and combinations thereof in an amount that is less than about 1000 μg, less than about 400 μg, less than about 300 μg, less than about 200 μg, or less than about 100 μg.

23. The method according to claim 11, wherein the compound is selected from (6S)-5-methyl, 10-formylTHF; (6S)-5-formyl, 10-formylTHF; derivatives thereof; and combinations thereof in an amount at least about 50% less than that recommended as a daily dietary allowance for a woman of childbearing age.

24. The method according to claim 11, wherein the compound or composition is administered in capsule, tablet, granule, or lozenge form.

25. The method according to claim 11, wherein said administering is parenteral.

26. The method according to claim 11, wherein said administering is oral.
